(19) United States
(12) Patent Application Publication
(10) Pub. No.: US 2024/0412355 A1
(43) Pub. Date: Dec. 12, 2024

(54) SYSTEMS AND METHODS FOR PERFUSION QUANTIFICATION

(71) Applicant: ACTIV Surgical, Inc., Boston, MA (US)

(72) Inventors: Yao Z Liu, Cranston, RI (US); Saloni Mehrotra, Boston, MA (US); Chibueze A Nwaiwu, Boston, MA (US); Vasiliy E Buharin, Boston, MA (US); John Oberlin, Boston, MA (US); Roman Stolyarov, Boston, MA (US); Peter CW Kim, Boston, MA (US); Emmanuel DeMaio, Watertown, MA (US); Mikael Marois, Boston, MA (US)

(21) Appl. No.: 18/694,849

(22) PCT Filed: Sep. 23, 2022
(86) PCT No.: PCT/US2022/044608

§ 371 Date: Mar. 22, 2024

Related U.S. Application Data
(60) Provisional application No. 63/248,361, filed on Sep. 24, 2021.

(51) Int. Cl. G06T 7/00

(52) U.S. Cl. CPC G06T 7/0012; G06T 2207/10064; G06T 2207/30104

(57) ABSTRACT

The present disclosure provides a method for quantifying perfusion. The method may comprise (a) obtaining at least one image of a surgical scene: (b) processing the at least one image to determine one or more perfusion characteristics associated with one or more reference regions in the surgical scene: and (c) determining one or more relative perfusion characteristics for a target region in the surgical scene based on the at least one image and the one or more perfusion characteristics associated with the one or more reference regions.

### CROSS-REFERENCE

This application claims the benefit of U.S. Provisional Application No. 63/248,361, filed Sep. 24, 2021, which application is incorporated herein by reference.

### BACKGROUND

Medical imaging technology may be used to capture images or video data of anatomical, physiological, pathological, and/or morphological features of a subject or patient during medical or surgical procedures. The images or video data captured may be processed and analyzed to provide medical practitioners (e.g., surgeons, medical operators, technicians, etc.) with a visualization of internal structures and processes within a patient or subject.

### SUMMARY

The present application relates generally to medical imaging (e.g., laser speckle contrast imaging), and more specifically, to processing of medical images to quantify perfusion in or near a tissue region of a subject. The systems and methods of the present disclosure may be implemented to measure perfusion and blood flow (e.g., in relative units). Such measurement may involve quantifying tissue perfusion in order to provide doctors and surgeons with a numerical display of perfusion that can aid a surgical procedure in or near a tissue region of a subject.

Perfusion assessment may be of importance when performing surgery as tissue healing is dependent on adequate perfusion and blood flow. Various methods exist for visualizing blood flow in surgery, in both open and minimally invasive form-factors. For example, dye-based methods using indocyanine green are common but may be limited by pharmacokinetics and subjective user interpretation. Dye-free methods may include, for example, visible spectroscopy, multispectral and hyperspectral imaging, and laser speckle contrast imaging.

Laser Speckle Contrast Imaging (LSCI) is an optical technique that uses laser light to illuminate a diffuse surface to produce a visual effect known as a speckle pattern. Coherent laser light may be applied to biologic tissues to generate a pattern of diffraction, known as “speckle”. When applied to red blood cells moving within blood vessels, laser light may be used to visualize blood flow by capturing speckle on a camera. At present, LSCI produces color heatmaps to reflect tissue perfusion and blood flow, where areas of higher flow appear on the higher end of the heatmap (which may be set as red in some systems) and areas of lower flow appear on the lower end of the heatmap (which may be set as blue in some systems). Further, measurement of tissue perfusion and blood flow through laser speckle contrast imaging may not be standardized or uniformly defined in commercially available imaging systems, which may instead display arbitrary speckle contrast units that cannot be easily interpreted by doctors or surgeons unfamiliar with or unaccustomed to those imaging systems. The systems and methods of the present disclosure may be implemented to visualize perfusion using LSCI by way of relative perfusion units (RPU) and/or one or more color heatmaps.

Many current methods of measuring tissue perfusion rely on defining blood flow or oxygenation. Oxygenation saturation may be quantified as ratio of oxygenated to deoxygenated hemoglobin based on the well-known extinction coefficient spectra of hemoglobin, as is done in multispectral and hyperspectral imaging. Flow may be measured in volume per unit time or distance per unit time (e.g., velocity) using doppler ultrasound, but this may represent a stand-alone value that does not take background tissue perfusion into account.

There is a dearth of standardized quantification or measurement of tissue perfusion or blood flow using laser speckle contrast imaging, in relative units using nearby tissue as reference. Putting perfusion units in context of background tissue, such as well-perfused and/or ischemic tissue, may allow for improved interpretability of the clinical significance of a given tissue's perfusion measurement. For example, knowing that tissue X measures at 50% of the perfusion of tissue Y may help surgeons to better judge the health of tissue X and may inform certain intraoperative decisions, such as whether to create an intestinal anastomosis. A method of standardizing perfusion quantification based on background tissue perfusion is herein described.

In an aspect, the present disclosure provides a method for quantifying perfusion. The method may comprise: (a) obtaining at least one image of a surgical scene; (b) processing the at least one image to determine one or more perfusion characteristics associated with one or more reference regions in the surgical scene; and (c) determining one or more relative perfusion characteristics for a target region in the surgical scene based at least in part on the at least one image and the one or more perfusion characteristics associated with the one or more reference regions.

In some embodiments, the method further comprises distinguishing an etiology of tissue ischemia as an inflow obstruction or an outflow obstruction based on the one or more relative perfusion characteristics. In some embodiments, the inflow obstruction comprises an arterial obstruction. In some embodiments, the outflow obstruction comprises a venous obstruction. In some embodiments, (b) comprises providing a linear relationship between the one or more reference regions and using the linear relationship to provide the one or more relative perfusion characteristics. In some embodiments, the linear relationship relates a laser speckle contrast value to the one or more relative perfusion characteristics.

In some embodiments, the method further comprises differentiating between arterial and venous obstructions. In some embodiments, the differentiating is based at least in part on pulsatility behavior referenced numbers. In some embodiments, the differentiating is based at least in part on one or more colormaps comprising laser speckle data. In some embodiments, the method further comprises providing real time guidance or medical inferences based on the differentiating. In some embodiments, the method further comprises using the differentiating to generate a guidance model or a classification model.

In some embodiments, the at least one image comprises at least one member selected from the group consisting of: a laser speckle image, a time-of-flight image, one or more multispectral images, or a fluorescence image. In some embodiments, the fluorescence image uses a fluorophore comprising indocyanine green, fluoresceine, or riboflavin. In some embodiments, the laser speckle image is a laser speckle contrast image. In some embodiments, the time-of-flight image is a doppler image.

In some embodiments, the at least one image comprises at least a laser speckle image and at least one image of a second image type, wherein the second image type is selected from the group consisting of: a time-of-flight image, one or more multispectral images, or a fluorescence image. In some embodiments, the method further comprises deriving one or more parameters from the second image type and generating an absolute perfusion model based on the one or more parameters.

In some embodiments, the method further comprises providing a comparative analysis between subjects. In some embodiments, the comparative analysis provides an indication of a predicted surgical outcome. In some embodiments, the method further comprises providing a trained classifier model based on the one or more parameters. In some embodiments, the trained classifier model is configured to provide an output comprising an absolute perfusion metric. In some embodiments, the trained classifier model is configured to provide a classification of a tissue as one of perfused, ischemic, watershed, or an unknown perfusion state. In some embodiments, the trained classifier model is a machine learning algorithm. In some embodiments, the providing comprises training the trained classifier based on a plurality of classified images.

In some embodiments, the method further comprises using one or more time-of-flight measurements to standardize perfusion quantification independent of camera positioning. In some embodiments, the method further comprises providing an imaging sensor configured to receive a plurality of light signals reflected from the surgical scene and to output the at least one image of the surgical scene. In some embodiments, the imaging sensor comprises: a first imaging unit configured for time of flight (TOF) imaging; and a second imaging unit configured for at least one of laser speckle imaging and fluorescence imaging. In some embodiments, imaging sensor comprises a third imaging unit configured for RGB imaging.

In an aspect, the present disclosure provides a system for medical imaging. The system may comprise: a processor comprising operably connected to a non-transitory computer readable storage medium with instructions stored thereon, wherein the processor is configured to implement the instructions to at least: (a) obtain at least one image of a surgical scene; (b) process the at least one image to determine one or more perfusion characteristics associated with one or more reference regions in the surgical scene; and (c) determine one or more relative perfusion characteristics for a target region in the surgical scene based at least in part on the at least one image and the one or more perfusion characteristics associated with the one or more reference regions.

In some embodiments, the processor is further configured to determine an etiology of tissue ischemia as an inflow obstruction or an outflow obstruction based on the one or more relative perfusion characteristics. In some embodiments, the inflow obstruction comprises an arterial obstruction. In some embodiments, the outflow obstruction comprises a venous obstruction. In some embodiments, at (b) the processor is further configured to provide a linear relationship between the one or more reference regions and using the linear relationship to provide the one or more relative perfusion characteristics. In some embodiments, the linear relationship relates a laser speckle contrast value to the one or more relative perfusion characteristics.

In some embodiments, the processor is further configured to differentiate between arterial and venous obstructions. In some embodiments, the differentiating is based at least in part on pulsatility behavior referenced numbers. In some embodiments, the differentiating is based at least in part on one or more colormaps comprising laser speckle data. In some embodiments, the processor is further configured to provide real time guidance or medical inferences based on the differentiating. In some embodiments, the processor is further configured to use the differentiating to generate a guidance model or a classification model.

In some embodiments, the at least one image comprises at least one member selected from the group consisting of: a laser speckle image, a time-of-flight image, one or more multispectral images, or a fluorescence image. In some embodiments, the fluorescence image uses a fluorophore comprising indocyanine green, fluoresceine, or riboflavin. In some embodiments, the laser speckle image is a laser speckle contrast image. In some embodiments, the time-of-flight image is a doppler image.

In some embodiments, the at least one image comprises at least a laser speckle image and at least one image of a second image type, wherein the second image type is selected from the group consisting of: a time-of-flight image, one or more multispectral images, or a fluorescence image. In some embodiments, the processor is further configured to derive one or more parameters from the second image type and generate an absolute perfusion model based on the one or more parameters.

In some embodiments, the processor is further configured to provide a comparative analysis between subjects. In some embodiments, the comparative analysis comprises an indication of a predicted surgical outcome. In some embodiments, the processor is further configured to provide a trained classifier model based on the one or more parameters. In some embodiments, the trained classifier model is configured to provide an output comprising an absolute perfusion metric. In some embodiments, the trained classifier model is configured to provide a classification of a tissue as one of perfused, ischemic, watershed, or an unknown perfusion state. In some embodiments, the trained classifier model is a machine learning algorithm. In some embodiments, the processor is further configured to train the trained classifier based on a plurality of classified images.

In some embodiments, the processor is further configured to use one or more time-of-flight measurements to standardize perfusion quantification independent of camera positioning. In some embodiments, the system further comprises an imaging sensor configured to receive a plurality of light signals reflected from the surgical scene and to output the at least one image of the surgical scene. In some embodiments, the imaging sensor comprises: a first imaging unit configured for time of flight (TOF) imaging; and a second imaging unit configured for at least one of laser speckle imaging and fluorescence imaging. In some embodiments, the imaging sensor comprises a third imaging unit configured for RGB imaging.

In another aspect, the present disclosure provides methods for differentiating the etiology of tissue ischemia as arterial or venous in nature, based on perfusion data.

In a further aspect, the present disclosure provides methods to correct for motion, distance, and angle artifacts in the context of laser speckle contrast imaging.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example image of a Perfused-Ischemic Gradient in a small bowel.

FIG. 2A is an image of a perfused region with no occlusion, e.g., experiencing substantially normal blood flow.

FIG. 2B is an image of an ischemic region, e.g., a region which does not have normal blood flow because flow is reduced, limited, or restricted.

FIG. 2C and FIG. 2D are images of partial and complete arterial occlusion, respectively.

FIG. 2E and FIG. 2F are images of partial and complete venous occlusion, respectively.

FIG. 3 is a flow chart of an example method for quantifying perfusion.

FIG. 4A schematically illustrates a system for medical imaging, in accordance with some embodiments.

FIG. 4B schematically illustrates a scope assembly, in accordance with some

embodiments.

FIG. 5 schematically illustrates a computer system that is programmed or otherwise configured to implement methods provided herein.

FIG. 6A shows a box scatter plot with X-axis showing perfused (x), watershed (y), and ischemic (z) bowel segments. Also shown are positive (p) and negative (n) controls.

FIG. 6B shows the regions x, y, z, p, and n in a representative porcine small intestine model.

FIG. 7 shows a scatter plot of relative perfusion units of bowel segments versus the mean femoral arterial pressure during progressive aortic occlusion.

FIG. 8 shows a scatter plot of relative perfusion units of bowel segments versus the mean femoral arterial pressure during progressive portal vein occlusion.

FIG. 9 shows a scatter plot of relative perfusion units of bowel segments with no arterial/venous occlusion vs progressive aortic occlusion vs progressive portal vein occlusion.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out.

The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

The term “real time” or “real-time,” as used interchangeably herein, generally refers to an event (e.g., an operation, a process, a method, a technique, a computation, a calculation, an analysis, a visualization, an optimization, etc.) that is performed using recently obtained (e.g., collected or received) data. In some cases, a real time event may be performed almost immediately or within a short enough time span, such as within at least 0.0001 millisecond (ms), 0.0005 ms, 0.001 ms, 0.005 ms, 0.01 ms, 0.05 ms, 0.1 ms, 0.5 ms, 1 ms, 5 ms, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 ms, 1 ms, 0.5 ms, 0.1 ms, 0.05ms, 0.01 ms, 0.005 ms, 0.001 ms, 0.0005 ms, 0.0001 ms, or less.

As used herein, the terms “subject” and “patient” are used interchangeably. As used herein, the terms “subject” and “subjects” refers to an animal (e.g., birds, reptiles, and mammals), a mammal including a primate (e.g., a monkey, chimpanzee, and a human) and a non-primate (e.g., a camel, donkey, zebra, cow, pig, horse, cat, dog, rat, and mouse). In certain embodiments, the mammal is 0 to 6 months old, 6 to 12 months old, 1 to 5 years old, 5 to 10years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100.

The devices, methods, and methods of use and manufacture as disclosed herein may be used to characterize a number of biological tissues to provide a variety of diagnostic information. A biological tissue may comprise a patient organ. Imaging devices disclosed herein may be disposed within a bodily cavity to characterize a patient tissue. A patient organ or bodily cavity may comprise for example: a muscle, a tendon, a ligament, a mouth, a tongue, a pharynx, an esophagus, a stomach, an intestine, an anus, a liver, a gallbladder, a pancreas, a nose, a larynx, a trachea, lungs, a kidneys, a bladder, a urethra, a uterus, a vagina, an ovary, a testicle, a prostate, a heart, an artery, a vein, a spleen, a gland, a brain, a spinal cord, a nerve, etc., to name a few. Imaging devices disclosed herein may be used in conjunctions with minimally invasive surgery, e.g., laparoscopic surgery. Imaging devices disclosed herein may include an endoscope, a laparoscope, etc.

### Overview

The present disclosure provides methods and systems for standardizing perfusion quantification based on background tissue perfusion. Such standardized perfusion quantification may provide numerical data on tissue perfusion or blood flow in relative units and based on perfusion characteristics of nearby tissue as a reference. The tissue perfusion or blood flow may be detectable or measurable using, for example, laser speckle contrast imaging. The methods disclosed herein may provide perfusion units in context of background tissue, such as well-perfused and/or ischemic tissue, thereby allowing for improved interpretability of the clinical significance of a given tissue's perfusion measurement. For example, knowing that tissue X measures at 50% of the perfusion of tissue Y may help surgeons to better judge the health of tissue X and may inform certain intraoperative decisions, such as whether to create an intestinal anastomosis.

The systems and methods of the present disclosure may be implemented to measure and quantify tissue perfusion and blood flow in relative terms using reference tissue. Such measurement and quantification may occur in real-time during a surgical procedure, using, for example, laser speckle technology. The systems and methods disclosed herein may also be used to differentiate between arterial (inflow obstruction) and venous (outflow obstruction) etiologies for tissue ischemia.

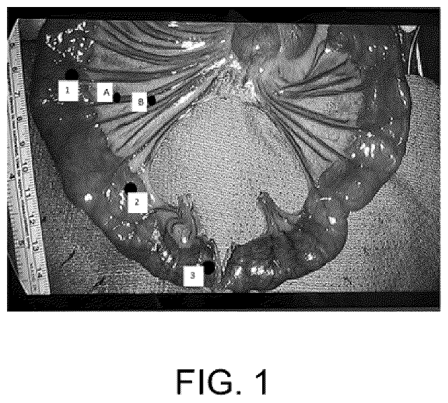

FIG. 1 schematically illustrates a Perfused-Ischemic Gradient in a small bowel. The small bowel may have different perfusion characteristics (e.g., perfused, marginal/watershed, ischemic) in different regions. The systems and methods of the present disclosure may utilize one or more controls to assess perfusion in a target region. The one or more controls may comprise, for example, a positive control associated with a mesenteric vessel, and a negative control associated with an avascular mesentery. In the illustrated example, various regions of the small bowel are labeled as follows: 1—perfused; 2—marginal/watershed; 3—ischemic; A—mesenteric vessel (positive control); and B-avascular mesentery (negative control).

A perfused region may be supplied with blood. An ischemic region may have blood flow that is restricted, limited, or reduced. For example, blood flow may be restricted, limited, or reduced temporarily or permanently. In some cases, a surgeon may intentionally limit blood flow to a region so as to limit subject bleeding during a procedure. A marginal/watershed region may be a region between a perfused region and an ischemic region. Blood flow in a marginal/watershed region may be partially restricted relative to blood flow absent the restriction. A mesenteric vessel may provide oxygenated blood to the intestines. The avascular mesentery may characterize a region which does not have a vessel present. These regions are of surgical relevance because they are regions where the mesentery may be safely divided, e.g., with reduced blood loss relative to division within the vascularized portion of the mesentery.

As shown in FIG. 1, ischemic region 3 is considerably darkened as compared to perfused region 1, due to ischemia caused by severing of the mesenteric vessels serving the region of the small bowel.

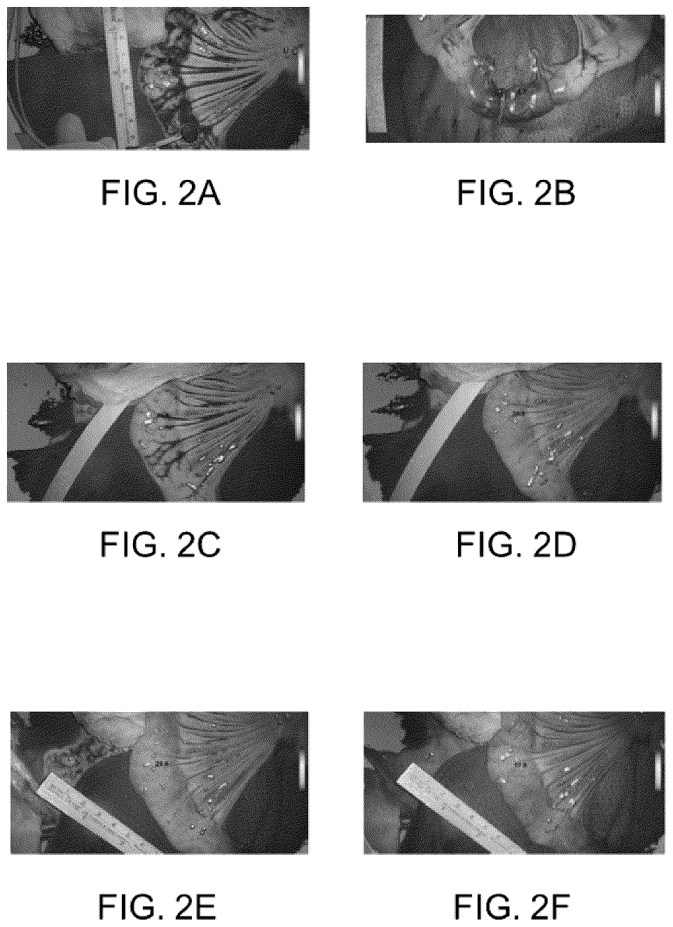

FIGS. 2A-2F illustrates an example sequence of images showing perfusion in a tissue region experiencing various states of occlusion through a laser speckle contrast image (LSCI) heatmap overlaid onto the RGB camera image, where tissue with more blood flow is darker (higher end of heatmap spectrum, on right side of screen) and tissue with less blood flow is lighter (lower end of heatmap spectrum).

FIG. 2A is an image of a perfused region with no occlusion, e.g., experiencing substantially normal blood flow. FIG. 2B is an image of an ischemic region, e.g., a region which does not have normal blood flow because flow is reduced, limited, or restricted. FIG. 2C and FIG. 2D are images of partial and complete arterial occlusion, respectively. In the example shown, the arterial occlusion is at the proximal aorta. FIG. 2E and FIG. 2F are images of partial and complete venous occlusion, respectively. In the example shown, the venous occlusion is at the portal vein. As shown, the LSCI images show differences based on whether the occlusion is arterial or venous; however, reduced flow can be observed under both types of occlusions.

### Laser Speckle Contrast Imaging

Precise and accurate intraoperative assessment of tissue perfusion, for example, at intestinal anastomoses, is a useful determinant of surgical outcomes. Laser Speckle Contrast Imaging (LSCI) is an emerging technology that allows for the visualization of tissue perfusion in a dye free, repeatable manner. LSCI may be used to detect and display real-time tissue perfusion/blood flow in a colormap with spatiotemporal precision and accuracy. The present disclosure provides methods and systems for quantifying perfusion measured using LSCI, correlating tissue perfusion colormaps, and detecting differential responses to arterial/venous occlusion. The present disclosure further provides a quantification function of LSCI correlating with tissue perfusion colormap.

The systems and methods of the present disclosure may be implemented to provide a laser speckle perfusion indicator. The laser speckle perfusion indicator may be controlled (e.g., moved or repositioned) by a user to indicate a region of interest that a user would like to further analyze for perfusion characteristics. The systems and methods disclosed herein may be implemented to provide relative perfusion measurements for the region indicated by the laser speckle perfusion indicator.

The systems and methods of the present disclosure may be implemented to provide a laser speckle perfusion image. In some cases, the present disclosure provides systems and methods for Laser Speckle Contrast Imaging (LSCI). LSCI is a non-scanning wide field-of-view optical technique utilized in a wide range of applications such as for imaging blood flow. When laser light illuminates a diffuse surface, the high coherence of the light produces a random granular effect known as speckle. Speckle patterns are generated on a target due to light interference which is spatially blurred due to the movement of scattering particles. Image frames containing the speckle patterns can be analyzed to compute dynamic and structural quantities of the target.

The present disclosure provides methods and systems for processing laser speckle images. In some cases, a series of frames F_1, F_2, . . . , F_N of a scene illuminated with laser light may be collected using a camera. The camera may include, for example, a universal serial bus (USB) camera that uses USB technology to transfer data. The coherence of the laser light causes a speckle pattern to appear on the scene. This speckle pattern may depend on the location of the observer and the intrinsic parameters of the camera. For example, two cameras at different locations may capture different speckle patterns, and two users observing the scene (camera to eye) may not agree on the location of speckles. If the object being imaged happens to be moving, then the speckle pattern on its surface may change from frame to frame, in a random “twinkling” which does not resemble a pattern flowing with the motion of the object and may not readily be “tracked.” By examining groups of neighboring pixels (either in space, or from frame to frame) and computing the mean (μ) and variance (σ\hat{ }2)of those neighboring pixels, the velocity of the object being imaged at each pixel can be computed as approximately (μ\hat{ }2/σ\hat{ }2). Detected motion can be due to physical motion of the object or due to blood flow in the underlying tissue.

The methods and systems disclosed herein may be implemented by deriving a statistical quantity (μ\hat{ }2/σ\hat{ }2) of each pixel. The quantity (μ\hat{ }2/σ\hat{ }2)may be related to the laser speckle contrast. These quantities may be estimated empirically.

For example, instead of estimating μ˜Σ_{i=1 . . . ,n} pᵢ, μ in the present disclosure can be estimated by μₜ=(1−α)pₜ+α*μₜ₋₁ Similarly, a sum of squares xₜⁱ=(1−α)pₜ²+α*xₜ₋₁ⁱ can be estimated. μ₂ and xₜⁱ may correspond to the “counts” at time t. σₜ²=xₜⁱ−μₜ². Consequently, (μ\hat{ }2/σ\hat{ }2)can be simplified into an expression that uses fewer division operations, thereby increasing computational performance.

The pulse of a patient may modulate the flow of blood and perfusion of tissue in a periodic way. This pulse can be detected from a whole image and used directly or used as the basis to synthesize a pure reference pulse signal of the appropriate frequency and phase. Flow which varies with the pulse signal may arise due to blood flow, while flow which does not vary with the signal may arise due to physical motion e.g., peristalsis, respiration, or camera motion.

The laser speckle signal may comprise a signal that is associated with a laser speckle pattern. The laser speckle pattern may comprise a pattern that is generated on a material when the material is exposed to (i.e., illuminated by) one or more laser light beams or pulses. The material may comprise a tissue region of a subject. The material may comprise a biological material. In some cases, the biological material may comprise a portion of an organ of a patient or an anatomical feature or structure within a patient's body. In some cases, the biological material may comprise a tissue or a surface of a tissue of the patient's body. The tissue may comprise epithelial tissue, connective tissue, organ tissue, and/or muscle tissue (e.g., skeletal muscle tissue, smooth muscle tissue, and/or cardiac muscle tissue).

The laser speckle pattern may be generated using at least one laser light source. The at least one laser light source may be configured to generate one or more laser light beams or pulses. The one or more laser beams or pulses may have a wavelength between about 400 nanometers (nm) and about 2500 nm, between about 700 nm and about 2500 nm, between about 700 and about 1500 nm. In some cases, the one or more laser beams or pulses may have a wavelength of about 808 nm, about 852nm, about 785 nm, etc. In some cases, the laser speckle pattern may be generated using a plurality of laser light sources configured to generate a plurality of laser beams or pulses having different wavelengths. The plurality of laser beams or pulses may have a wavelength between about 400 nanometers (nm) and about 2500 nm, between about 700 nm and about 2500 nm, between about 700 and about 1500 nm. In some cases, the plurality laser beams or pulses may have a wavelength of about 808 nm, about 852 nm, about 785 nm, etc. In some cases, the at least one laser light source may comprise a coherent light source, such as a laser diode. In some cases, the at least one laser light source may be configured to generate light in a near-infrared spectrum range. The light in the near-infrared spectrum range may have a wavelength between about 700 nm to about 2500 nm, between about 700 and about 1500 nm. In some cases, the near-infrared light may comprise a wavelength of about 980 nm, about 808 nm, about 852nm, about 785 nm, etc.

The speckle patterns may be produced due to an interference of light beams or light rays that is caused by a coherent light source (e.g., a laser) when illuminating a target site or target region (e.g., sample, tissue, organ in human body, etc.). When the light beams or light rays impinge the target site/region (e.g., a tissue surface), they may be scattered and/or reflected from different portions of the target site/region or different features within the target site/region. Due to variations in a structure or a topology of the target site/region or variations in a position or a movement of one or more scattering particles (e.g., biological materials) in or near the target site/region, the light beams or light rays may travel different distances such that the scattered light beams or light rays are subjected to random variations in phase and/or amplitude. This may result in patterns of constructive and/or destructive interference, which may change over time depending on a position of different features and/or a movement of one or more scattering particles. The scattered light may produce a randomly varying intensity pattern known as a speckle pattern. If the scattering particles are moving, this may cause fluctuations in the interference, which may appear as intensity variations. The temporal and spatial statistics of such speckle patterns may provide information about a motion of one or more underlying objects, features, or biological materials being imaged.

One or more imaging devices may be used to image the speckle patterns. The one or more imaging devices may comprise a photodetector that is configured to receive scattered light that is reflected from different portions of the target site/region or different features within the target site/region. The laser speckle patterns may be obtained using one or more imaging devices. In some cases, the laser speckle patterns may be obtained over a plurality of frames as the plurality of frames are being received or processed in real time by the one or more imaging devices. The one or more imaging devices may comprise a camera, a video camera, a Red Green Blue Depth (RGB-D) camera, an infrared camera, a near infrared camera, a charge coupled device (CCD) image sensor, a complementary metal oxide semiconductor (CMOS) image sensor, a linear image sensor, an array silicon-type image sensor, and/or an InGaAs (Indium gallium arsenide) sensor. The one or more imaging devices may be configured to capture an image frame or a sequence of image frames. The image frame or the sequence of image frames may comprise one or more laser speckle patterns that are generated on a tissue surface using the at least one laser light source.

The image frame or the sequence of image frames may be provided to an image processing module. The image processing module may be configured to derive one or more laser speckle signals from the image frame or the sequence of image frames captured using the one or more imaging devices. In some cases, the image processing module may be configured to process the captured speckle images to convert the intensity of the scattered light within the image frame or the sequence of images frames into a digital signal. The digital signal may correspond to a laser speckle signal as described herein. In some cases, the digital signal may be used to generate one or more laser speckle contrast images and/or provide information about a biological process within a tissue region of the subject's body. In some cases, the biological process may comprise a movement of a biological material or a flow of a biological fluid within or near the tissue region.

The image processing module may be configured to process one or more raw speckle images comprising one or more speckle patterns to generate laser speckle contrast images. The laser speckle contrast images may comprise information on a speckle contrast associated with one or more features of the laser speckle patterns within the raw speckle images. The speckle contrast may comprise a measure of local spatial contrast values associated with the speckle patterns. The speckle contrast may be a function of a ratio between the standard deviation of the intensity of the scattered light and the mean of the intensity of the scattered light. If there is a lot of movement in the speckle pattern, blurring of the speckles in the speckle pattern may increase, and the standard deviation of the intensity may decrease. Consequently, the speckle contrast may be lower.

In some cases, the laser speckles images, the laser speckle patterns, and/or the laser speckle contrast images may be processed to obtain fluid flow information for one or more fluids that are moving and/or present in or near the tissue region. In some cases, the fluid may comprise blood, sweat, semen, saliva, pus, urine, air, mucus, milk, bile, a hormone, and/or any combination thereof. In some cases, a fluid flow rate within the target tissue may be determined by a contrast map or contrast image generated using the captured speckle images and/or one or more laser speckle signals derived from the captured speckle images.

The biological material may be within the subject's body. In some cases, the biological material may be a part of the subject's body. In some cases, the biological material may comprise a tissue. The tissue may comprise epithelial tissue, connective tissue, organ tissue, and/or muscle tissue (e.g., skeletal muscle tissue, smooth muscle tissue, and/or cardiac muscle tissue). In some cases, the biological material may comprise the subject's skin. In some cases, the biological material may comprise a fluid. The fluid may comprise blood, lymph, tissue fluid, milk, saliva, semen, bile, an intracellular fluid, an extracellular fluid, an intravascular fluid, an interstitial fluid, a lymphatic fluid, and/or a transcellular fluid.

### Hyperspectral Imaging

The present disclosure also provides methods and systems for laser speckle spectral deconvolution. Spectral deconvolution can be applied to the speckle maps developed under different wavelengths. This technique may be referred to herein as “hyperspectral.” The methods and systems disclosed herein may be implemented using any number of wavelengths. The methods and systems disclosed herein may be implemented using any one or more aspects of general spectroscopy. The methods and systems disclosed herein may be implemented for the purpose of hemoglobin (Hb) versus Parenchyma concentration determination. The methods and systems disclosed herein may be implemented to evaluate oxygenation (SP02) from speckle under two or more wavelengths.

The present disclosure provides systems and methods for multispectral and/or hyperspectral imaging. As used herein, multispectral imaging may refer to spectral imaging using a plurality of discrete wavelength bands. As used herein, hyperspectral imaging may refer to imaging a plurality of spectral wavelength bands over a continuous spectral range. In some cases, hyperspectral imaging may comprise capturing intensity information at each pixel coordinate across many wavelength bands other than the standard red, blue, and green (RBG) colors, thereby providing increased insight into tissue oxygenation and blood perfusion. The absorption, reflection, and scattering of light incident on a biological material or a physiological feature may depend on the chemical properties of the material or feature as well as the imaging wavelength used, and images obtained from additional spectra as in hyperspectral imaging can include information on compositions, concentrations, or other properties or characteristics of a surgical scene that is difficult to visualize using standard RGB imaging or the human eye.

The present disclosure also provides methods and systems for simultaneous multi-band speckle imaging. The hemoglobin (Hb) and blood oxyhemoglobin (HbO2) absorption spectra intersect at points known as isosbestic points. There is such a point near 808 nm. Speckle imaging at such a point would be theoretically agnostic to oxygenation and therefore should respond on the basis of flow alone. Thus, small veins and arteries of similar size and flow should appear the same (since structurally these vesicles are more similar than larger such vessels), and un-perfused tissue will not be biased by remaining levels of oxygen, which will change over time. By simultaneously illuminating in 785 nm and 852 nm with at a chosen intensity ratio, a scene can be imaged while maintaining invariance across Hb and HbO2. This can provide the benefit of imaging under an isosbestic point even though an optical system may not support a particular wavelength due to the need to block that wavelength which may be used for indocyanine green (ICG) fluorescence imaging excitation.

### Fluorescence Imaging

In some examples, diagnostic tools, such as fluorescent dye-based angiography (e.g., indocyanine green (ICG) angiography) may be used in conjunction to provide visualization of some complex anatomical or critical structures. ICG angiography, however, may be costly in terms of resources and time (e.g., ICG dyes may take several minutes to 24 hours to reach a target site), limited in accuracy (e.g., dyes may dissipate to non-target sites during surgery), induce allergic reactions in some patients, and/or lack real-time visualization capabilities. In addition, the use of imaging tools alone for endoscopy and angiography may lead to further surgical complications, for example because of prolonged surgical time or increased chance of contamination.

While ICG is disclosed herein other dyes such as fluoresceine or riboflavin may be used. Indocyanine green (ICG) is a water-soluble cyanine dye which shows fluorescence in the near-infrared range, with peak spectral absorption of about 790 nm in blood. In some cases, ICG may be introduced into the blood stream. The dye may then flow into areas that experience blood flow. Upon excitation in the area of interest, the ICG will fluoresce where it is present. The near IR fluorescence can be measured on a near-IR sensitive detector. Riboflavin, also known as Vitamin B2, may also be used for fluorescence imaging. Riboflavin fluoresces yellow-green when exposed to light. Riboflavin may offer certain advantages over ICG. For example, riboflavin may flush from the tissue more quickly (may not stain the tissue as resiliently) than ICG. The relatively quick removal of riboflavin from the tissue than ICG may allow for more responsive measurements of vessel occlusion.

In some cases, the present disclosure provides an imaging device that is configured to capture and display fluorescence data. In some cases, the present disclosure provides an imaging device that is configured to capture and display both fluorescence images (e.g., ICG, riboflavin, fluoresceine, etc.) and LSCI (laser speckle contrast images) in surgery (e.g., minimally invasive surgery). As discussed herein, laser speckle contrast imaging may utilize coherent laser light to detect red blood cell motion, displayed with real-time perfusion color heat maps.

The imaging module and light engine may attach to a standard laparoscopic camera and endoscope. LSCI may display blood flow as color heatmaps (red/warm colors indicate more perfusion and blue/cool colors less perfusion). An investigative mode includes quantification of perfusion signals in relative units.

### Time of Flight

The present disclosure provides methods to correct for motion, distance, and angle artifacts in laser speckle contrast imaging within an endoscopic/laparoscopic form factor. The method may comprise obtaining one or more time of flight (TOF) depth measurements for a tissue region. The one or more TOF depth measurements may be used to estimate a position, an orientation, and/or a motion of a scope (relative to the surgical scene) based on the TOF depth measurements. The TOF depth measurements may be used to correct for one or more artifacts in a laser speckle contrast image based on the TOF depth measurements or based on one or more inferences derived from the TOF depth measurements (e.g., relative position, orientation, and/or motion of a scope in relation to the surgical scene). The one or more artifacts may be, for example, errors or inconsistencies in the laser speckle contrast image that are caused by scope motion and/or variations in scope distance or scope angle.

The present disclosure provides systems and methods for time of flight (TOF) medical imaging. The terms “time of flight,” “time-of-flight,” “ToF,” or “TOF,” as used interchangeably herein, may generally refer to one or more measurements of a time taken by an object, a particle, or a wave to travel a distance through a medium (e.g., fluid, such as a liquid or gas). Examples of the wave may include acoustic wave and electromagnetic radiation. Example acoustic data may include doppler data. The time measurement(s) may be used to establish a velocity and/or a path length of the object, particle, or wave. In some cases, time of flight may refer to the time required for emitted electromagnetic radiation to travel from a source of the electronic radiation to a sensor (e.g., a camera). In some cases, a time-of-flight measurement may correspond to the time required for the emitted electromagnetic radiation to reach a target tissue. Alternatively, a time-of-flight measurement may correspond to the time required for the emitted electromagnetic radiation to reach a target tissue and to be directed or re-directed (e.g., reflected) to a sensor. Such sensor, which may comprise a TOF sensor, may be adjacent to the source of the emitted electromagnetic radiation, or may be at a different location than the source. In some cases, a camera or an imaging sensor may be used to determine a time of flight based on a phase shift of emitted and received signal (e.g., electromagnetic radiation). Examples of time-of-flight cameras may include, but are not limited to, radio frequency (RF)-modulated light sources with phase detectors (e.g., Photonic Mixer Devices (PMD), Swiss Ranger™, Canesta Vision™), range gated imagers (e.g., ZCam™), and/or direct time-of-flight imagers (e.g., light detection and ranging (LIDAR)).

In some cases, the TOF sensor may be positioned along a common beam path of the plurality of light beams or light pulses reflected from the surgical scene. The common beam path may be disposed between the surgical scene and an optical element that can be used to split the plurality of light beams or light pulses into different sets of light signals. In some cases, the plurality of light beams or light pulses reflected from the surgical scene may be split into (i) a first set of light signals corresponding to the TOF light and (ii) a second set of light signals corresponding to white light, laser speckle light, and/or fluorescence excitation light. The first set of light signals may have a beam path that is different than that of the second set of light signals and/or the plurality of light beams or light pulses reflected from the surgical scene. In such cases, the TOF sensor may be positioned along a discrete beam path of the first set of light signals that is downstream of the optical element.

In some cases, TOF measurements may be used to standardize relative perfusion units (RPU) in a surgical scene regardless of location of camera and laser speckle perfusion indicator. One exemplary method of applying TOF to standardize RPU includes measuring/displaying RPU on tissue X with camera at location 1, then moving camera to location 2 (with different distance/angle/motion artifact relative to tissue X) and measuring/displaying the same RPU value on tissue X compared to camera at location 1.

In some cases, perfusion information (e.g., as represented using RPU) may be distance normalized using one or more time of flight measurements. Such distance normalization may provide increased objectivity for surgeons viewing and interpreting the perfusion information as a position and/or an orientation of the camera and/or laser speckle perfusion indicator changes.

### Combined Sensor

In some cases, an imaging device that captures and displays both ICG and LSCI in minimally invasive surgery may be used to implement the methods and systems of the present disclosure. In some cases, the imaging device may be used to perform a method for perfusion quantification, which method may involve measuring relative perfusion units (RPU) using reference areas of normally perfused and ischemic tissue. Such method may be implemented using a perfusion quantification algorithm that takes into account reference areas of normally perfused and/or ischemic tissue.

In some cases, a single imaging sensor may be used for multiple types of imaging (e.g., any combination of fluorescence imaging, TOF depth imaging, laser speckle imaging, and/or RGB imaging). In some cases, a single imaging sensor may be used for imaging based on multiple ranges of wavelengths, each of which may be specialized for a particular type of imaging or for imaging of a particular type of biological material or physiology.

In some cases, the one or more imaging sensors may comprise a multispectral imaging sensor and/or a hyperspectral imaging sensor. The multispectral imaging sensor and/or the hyperspectral imaging sensor may comprise, for example, a mosaic sensor. The mosaic sensor may be configured for imaging in a plurality of different wavelengths. The plurality of different wavelengths may lie in the visible light spectrum, the infrared light spectrum, the near infrared light spectrum, the short-wave infrared spectrum, the mid wave infrared spectrum, and/or the long wave infrared spectrum. In any of the embodiments described herein, the mosaic sensor may be configured for imaging in a plurality of different wavelength bands. In some cases, the mosaic sensor may comprise a plurality of cavities having different heights, which may enable the capture of different spectral wavelengths without requiring a separate optical element (e.g., one or more filters). The different spectral wavelengths may be registered at different pixels or sub-pixels of the imaging sensor, as described in greater detail below. In some cases, the pixels or sub-pixels of the imaging sensor may be capable of generating imaging data associated with multiple different wavelengths or spectral ranges.

In some cases, the imaging sensors described herein may comprise an imaging sensor configured for fluorescence imaging and at least one of RGB imaging, laser speckle imaging, and TOF imaging. In some cases, the imaging sensor may be configured for fluorescence imaging and at least one of RGB imaging, perfusion imaging, and TOF imaging. In any of the embodiments described herein, the imaging sensors may be configured to see and register non-fluorescent light.

In some cases, the imaging sensors may be configured to capture fluorescence signals and laser speckle signals during alternating or different temporal slots. For example, the imaging sensor may capture fluorescence signals at a first-time instance, laser speckle signals at a second time instance, fluorescence signals at a third time instance, laser speckle signals at a fourth time instance, and so on. The imaging sensor may be configured to capture a plurality of different types of optical signals at different times. The optical signals may comprise a fluorescence signal, a TOF depth signal, an RGB signal, and/or a laser speckle signal.

In other cases, the imaging sensor may be configured to simultaneously capture fluorescence signals and laser speckle signals to generate one or more medical images comprising a plurality of spatial regions. The plurality of spatial regions may correspond to different imaging modalities. For example, a first spatial region of the one or more medical images may comprise a fluorescence image based on fluorescence measurements, and a second spatial region of the one or more medical images may comprise an image based on one or more of laser speckle signals, white light or RGB signals, and TOF depth measurements.

### Relative Perfusion

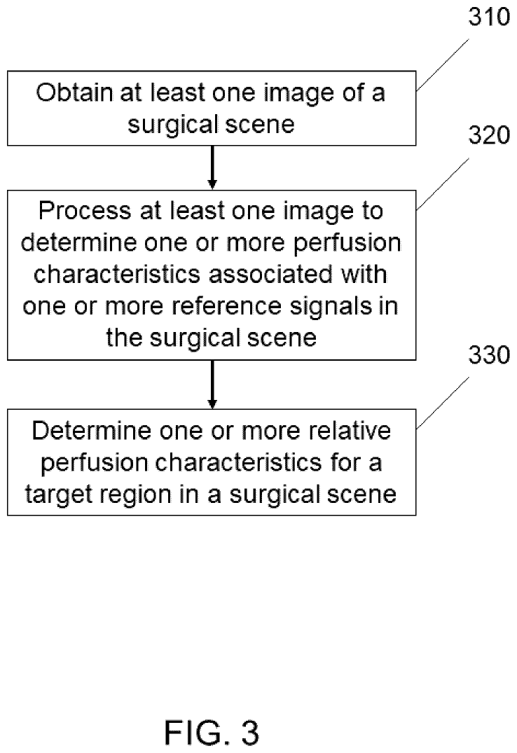

FIG. 3 is a flowchart of a method for quantifying perfusion. The method may be implemented by a processor, for example, computer system 501 described elsewhere herein. In some cases, methods of quantifying tissue perfusion and blood flow (e.g., using laser speckle technology, hyperspectral imaging, fluorescence imaging) may involve relative quantification of tissue perfusion. The relative perfusion may be quantified using reference tissue, for example, within an endoscopic/laparoscopic field of view as a standard for varying degrees of perfusion. For example, a reference tissue may be a positive control (e.g., a mesentery vessel) or a negative control (e.g., avascular mesentery tissue).

At an operation 310, the method may comprise obtaining at least one image of a surgical scene. In some examples, perfusion may be measured using relative laser speckle perfusion units on a tissue of interest; however, other images of a surgical scene may be employed instead of or in combination with laser speckle. For example, the at least one image comprises at least one member selected from the group consisting of: a laser speckle image, a time-of-flight image, one or more multispectral images, or a fluorescence image. In some cases, the fluorescence image is an indocyanine green image. In some cases, the laser speckle image is a laser speckle contrast image. In some cases, the time-of-flight image is a doppler image.

At an operation 320, the method may comprise processing the at least one image to determine one or more perfusion characteristics associated with one or more reference regions in the surgical scene. For example, laser speckle contrast may be associated with perfusion in a laser speckle images. In another example, fluorescence may be associated with perfusion in an ICG images.

A reference region may be a control, for example, a negative or a positive control, as described herein with respect to FIG. 1. In some examples, Tissue X, which may be “well-perfused” or “healthy” tissue, may be assigned a reference value of 100% by pointing a laser speckle perfusion indicator at Tissue X and registering its laser speckle perfusion units as 100% within the processing system.

The laser speckle perfusion indicator may be, for example, a user-provided or user-controlled indicator that can be used to define a boundary or a region in which perfusion is to be measured. The laser speckle perfusion indicator may be controlled using an input device (e.g., a polygon on a laparoscopic imaging camera display, a mouse, a trackpad, a touch screen, or any other device that is configured to detect an input and control a size or a position of the boundary or region in which perfusion is to be measured, based on the input). In some cases, perfusion may be measured by pointing an indicator (e.g., a circle, a square, a polygon, etc.) that is centered on a laparoscopic image display, at a tissue of interest. Perfusion units may be averaged for a region (e.g., a square region, a round region, a region of fit to the anatomy, etc.) of tissue immediately outside, and/or inclusive of, the indicator. The size and/or the shape of the perfusion indicator may be adjusted based on operator preference or based on the needs of the surgical procedure.

At an operation 320, a method may comprise determining one or more relative perfusion characteristics for a target region in the surgical scene based at least in part on the at least one image and the one or more perfusion characteristics associated with the one or more reference regions. In some cases, a Tissue Y, which may be “ischemic” or tissue that is otherwise known to have less flow than tissue X, may be assigned a reference value of Y% (where Y is between 0% and 100%) by pointing the laser speckle perfusion indicator at Tissue Y and registering its laser speckle perfusion units as Y% within the processing system. In some cases, completely ischemic and devascularized tissue may be represented as 0%. In some cases, less perfused but not completely ischemic tissue may be assigned a threshold value of Y% to compare to tissue X.

In some cases, a doctor or a surgeon may be interested in measuring perfusion in another point of interest, for example, Tissue Z. In some cases, the perfusion for a point of interest, Tissue Z, may be calculated and displayed as Z% of relative laser speckle perfusion units on a scale of Y% to 100% based on its comparison to tissue Y (being Y%) and tissue X (being 100%).

Relative perfusion may be computed according to the following equation: Z^{percent}=(Z^{relative laser speckle perfusion units}−Y^{relative laser speckle perfusion units})/(X^{relative laser speckle perfusion units}−Y^{relative laser speckle perfusion units}). For example, if Tissue X (“well-perfused” standard), Y (“ischemic” standard”), and Z (tissue of interest) demonstrate relative laser speckle perfusion units of 150, 25, and 75, respectively, the relative quantification of tissue Z may be calculated as: Z^{percent}=(75−25)/(150−25)=40%. The relative quantification of tissue Z (represented by Zpercent) may then be displayed to a doctor or surgeon to provide additional information on the perfusion characteristics of a target area relative to one or more reference areas with known perfusion characteristics.

In some case, a method quantifying perfusion further comprises providing a linear relationship between the one or more reference regions and using the linear relationship to provide the one or more relative perfusion characteristics. The relationship between blood flow and the LSCI data may be substantially linear. For example, the contrast ratio (μ\hat{ }2/σ\hat{ }2) may be linear with flow. By contrast a metric based on the relation (1−σ\hat{ }2)/μ\hat{ }2 may increase monotonically with flow but may not comprise a linear relationship with flow. Accordingly, the linear relationship may relate a laser speckle contrast value to the one or more relative perfusion characteristics. The linear relationship between speckle contrast and flow may allow for linear interpolation of the relative flow between controls without departing from a physically relevant measure of flow.

While laser speckle imaging is disclosed in the above example, relative perfusion may be measured from the relative fluorescence at various locations in the surgical scene. For example, the relative perfusion unit calculation may be used to measure the time kinetics of ICG data. ICG data may not be as responsive with time to changes in occlusion. For example, because the blood is not flowing, residual dye may collect in a vessel after occlusion. Laser speckle data may improve upon ICG imaging at least in part because laser speckle data directly measures tissue motion. Even with the slower time dynamics of ICG imaging, the ICG data may exhibit a slow reduction in signal after clamping followed by a fast rise in signal after removal of cause of the occlusion. Relative perfusion data may be used to determine quantitatively changes in flow over time. The relationship between ICG signal and flow may not be linear. Instead, relative flow may be log-linear with fluorescence signal.

In some examples, relative perfusion may be measured from the relative speckle signal at various wavelengths and various locations in the surgical scene. For example, by using a plurality of wavelengths relative perfusion may be corrected for different tissue types. For example, the tissue may be corrected for contributions from fat. For example, measurements at various wavelengths may be used to provide simultaneous information about blood oxygenation. The blood oxygenation may be used to correct the relative perfusion.

### Arterial vs Venous Obstructions

The present disclosure provides methods for distinguishing etiology of tissue ischemia as arterial (inflow obstruction) or venous (outflow obstruction). Laser speckle contrast imaging may generate relative laser speckle perfusion units, and arterial/venous obstructions may generate characteristically different mathematical patterns of perfusion unit variation when benchmarked against an objective clinical standard. For example, relative laser speckle perfusion units used on tissue with arterial ischemia may demonstrate or exhibit a linear relationship to mean arterial pressure, whereas a non-linear (at times exponential) relationship to mean arterial pressure may be demonstrated for tissue with venous ischemia.

In some cases, a method of quantifying perfusion may comprise distinguishing an etiology of tissue ischemia as an inflow obstruction or an outflow obstruction based on the one or more relative perfusion characteristics. The inflow obstruction may comprise an arterial obstruction. The outflow obstruction may comprise a venous obstruction.

In some cases, a method of quantifying perfusion may comprise differentiating between arterial and venous obstructions. For example, the differentiating may be based at least in part on pulsatility behavior referenced numbers. For example, the differentiating may be based at least in part on one or more colormaps comprising laser speckle data. For example, the method may comprise providing real time guidance or medical inferences based on the differentiating. In some cases, the method of quantifying perfusion comprises using the differentiating to generate a guidance model or a classification model.

In some cases, tissue ischemia may be distinguished as arterial (inflow obstruction) or venous (outflow obstruction) based on pulsatility. Pulsatility may be assessed either through subjective visual inspection or objective clinical measures. On visual inspection, tissue experiencing venous obstruction may appear significantly less pulsatile. Tissue experiencing arterial obstruction may not appear to lose pulsatility to the same degree. Using an objective measure, pulsatility may be measured by intensity and color demonstrated on LSCI perfusion colormap. Pulsatility may also be quantified using change in RPU measurements from baseline, arterial pulse pressure and/or pulsatility index. Pulse pressure (PP) may refer to the difference between the maximum and minimum pressure. Pulsatility index (PI) may refer to the difference between peak systolic and minimum diastolic blood flow velocity, divided by the mean velocity during a cardiac cycle. In some cases, pulsatility (both cyclical (diastolic and systolic) cardiac and respiratory) and/or dampening of pulsatility may be used to quantitatively differentiate between arterial and venous obstructions which may contribute to tissue ischemia.

### Absolute Perfusion

Systems and methods disclosed herein may be used to develop an absolute quantification of tissue perfusion. For example, in a relative perfusion quantification, relative perfusion may be derived based on a comparison to another speckle signal in the surgical scene.

An absolute perfusion quantification may relate to an output of a quantitative value of flow rate that is not based on other flow signals in the scene. From absolute prefusion, the value of blood flow may be converted to units of velocity or blood pressure. To derive a quantitative value that approximates the actual flow rate, multiple variables may need to be extracted and considered in the computation. For example, the collected speckle signal is affected at least in part by the following factors: the underlying flow rate, optical properties of the tissue, the depth of the flow below the tissue, the concentration of the flowing particles, and the camera gain. In some cases, a single absolute perfusion value may be determined, and the relative perfusion model may be used to determine absolute perfusion at other points in the surgical scene.

Having access to a value of absolute quantification as it is outlined here provides a multitude of advantages, such as the ability to compare flow values between patients, and even across modalities that detect velocity or blood pressure values in a different manner (e.g., using Doppler ultrasound or a blood pressure monitor).

In some cases, a method of quantifying perfusion may comprise collecting at least a laser speckle image and at least one image of a second image type. For example, the second image type may be selected from the group consisting of: a time-of-flight image, one or more multispectral images, or a fluorescence image. In some cases, one or more parameters may be derived from the second image type. Using the one or more parameters, an absolute perfusion model based on the one or more parameters may be generated.

In some cases, flow rate may be derived as disclosed herein, for example, from laser speckle contrast. The optical properties of flowing blood and tissue covering the vessel may include the absorption or the scattering or both from the tissue. A hyperspectral sensor such as a mosaic sensor disclosed herein may allow the collection of absorption spectra. Absorption spectra may be processed to derive a value of optical properties at each pixel. The amount of speckle may be affected by a depth of flow (e.g., how much tissue is covering the flow). Depth may be measured, for example, by time-of-flight imaging, as disclosed herein. In some cases, depth may be approximated using multiple wavelengths of known penetration depths. Date using multiple wavelengths of known penetrations depths may be measured using a mosaic hyperspectral camera. The concentration of flowing particles may affect the amount of speckle signal. For blood, example, the concentration of flowing particles may be the concentration of blood cells. In some cases, the concentration of flowing particles may be approximated using known ranges of blood cell concentrations in humans. The camera gain may affect the amount of speckle signal. The gain setting of the camera at the time of acquisition may be collected from the imaging sensor.

A model of absolute perfusion may be constructed using one or more model parameters. One or more model parameters may include one or more of the underlying flow rate, optical properties of the tissue, the depth of the flow below the tissue, the concentration of the flowing particles, and the camera gain. From the one or more parameters, a model of absolute perfusion may be derived.

In some cases, the one or more model parameters may be used to create a multiparameter fit based on known absolute perfusion data. For example, a dataset including absolute perfusion as a function of any combination of the underlying flow rate, optical properties of the tissue, the depth of the flow below the tissue, the concentration of the flowing particles, and the camera gain may be used to develop a multi axis fit. From the fit, a function relating absolute perfusion to the one or more parameters may be derived. From this function, the measured speckle parameters for an example patient measurement may be related to absolute perfusion.

In some cases, a method of quantifying perfusion may comprises providing a trained classifier model based on the one or more parameters. For example, the trained classifier model may provide an output comprising an absolute perfusion metric. For example, the trained classifier model may provide a classification of a tissue as one of perfused, ischemic, watershed, or an unknown perfusion state. The trained classifier may not rely on an explicit fitting function relating the one or more parameters to an absolute perfusion metric. Instead, the trained classifier may be used to find the most predictive variables and to derive an output without a detailed understanding of the underlying functional relationship between the parameters. Further, in some cases, a quantified value of the perfusion may not be necessary, and, instead, an indication of a state of the tissue may be sufficient. For example, a qualitative indication of the tissue state may allow for physician guidance when a quantified value of perfusion is not required.

In some cases, one or more parameters are used to provide a comparative analysis between subjects. For example, the comparative analysis provides an indication of a predicted surgical outcome. In the case of a trained classifier, the classifier model may be used to group cases together. For example, patients with think tissue covering the flowing vessels may grouped together.

In some cases, the trained classifier model is a machine learning algorithm. A machine learning algorithm may be particularly helpful in the case where speckle images have a direct physical relationship to the underlying flow buy may be variable based on larger number of underlying parameters. For example, the machine learning algorithm may be trained based at least in part upon a plurality of classified images. For example, the machine learning algorithm may comprise one or more of linear regressions, logistic regressions, classification and regression tree algorithms, support vector machines (SVMs), naive Bayes, K-nearest neighbors, random forest algorithms, boosted algorithms such as XGBoost and LightGBM, neural networks, convolutional neural networks, and recurrent neural networks. The machine learning algorithm may be a supervised learning algorithm, an unsupervised learning algorithm, or a semi-supervised learning algorithm.

Machine learning algorithms may be used in order to make predictions using a set of parameters. One class of machine learning algorithms, artificial neural networks (ANNs), may comprise a portion of the classifier model. For example, feedforward neural networks (such as convolutional neural networks or CNNs) and recurrent neural networks (RNNs) may be used. A neural network binary classifier may be trained by comparing predictions made by its underlying machine learning model to a ground truth. An error function calculates a discrepancy between the predicted value and the ground truth, and this error is iteratively backpropagated through the neural network over multiple cycles, or epochs, in order to change a set of weights that influence the value of the predicted output. Training ceases when the predicted value meets a convergence condition, such as obtaining a small magnitude of calculated error. Multiple layers of neural networks may be employed, creating a deep neural network. Using a deep neural network may increase the predictive power of a neural network algorithm.

Additional machine learning algorithms and statistical models may be used in order to obtain insights from the parameters disclosed herein. Additional machine learning methods that may be used are logistic regressions, classification and regression tree algorithms, support vector machines (SVMs), naive Bayes, K-nearest neighbors, and random forest algorithms. These algorithms may be used for many different tasks, including data classification, clustering, density estimation, or dimensionality reduction. Machine learning algorithms may be used for active learning, supervised learning, unsupervised learning, or semi-supervised learning tasks. In this disclosure, various statistical, machine learning, or deep learning algorithms may be used to generate an output based on the set of parameters.

A machine learning algorithm may use a supervised learning approach. In supervised learning, the algorithm can generate a function or model from training data. The training data can be labeled. The training data may include metadata associated therewith. Each training example of the training data may be a pair consisting of at least an input object and a desired output value. A supervised learning algorithm may require the user to determine one or more control parameters. These parameters can be adjusted by optimizing performance on a subset, for example a validation set, of the training data. After parameter adjustment and learning, the performance of the resulting function/model can be measured on a test set that may be separate from the training set. Regression methods can be used in supervised learning approaches.

In some embodiments, the supervised machine learning algorithms can include but not being limited to neural networks, support vector machines, nearest neighbor interpolators, decision trees, boosted decision stump, boosted version of such algorithms, derivatives versions of such algorithms, or their combinations. In some embodiments, the machine learning algorithms can include one or more of: a Bayesian model, decision graphs, inductive logic programming, Gaussian process regression, genetic programming, kernel estimators, minimum message length, multilinear subspace learning, naive Bayes classifier, maximum entropy classifier, conditional random field, minimum complexity machines, random forests, ensembles of classifiers, and a multicriteria classification algorithm.

A machine learning algorithm may use a semi-supervised learning approach. Semi-supervised learning can combine both labeled and unlabeled data to generate an appropriate function or classifier.

In some embodiments, a machine learning algorithm may use an unsupervised learning approach. In unsupervised learning, the algorithm may generate a function/model to describe hidden structures from unlabeled data (i.e., a classification or categorization that cannot be directed observed or computed). Since the examples given to the learner are unlabeled, there is no evaluation of the accuracy of the structure that is output by the relevant algorithm. Approaches to unsupervised learning include: clustering, anomaly detection, and neural networks.

A machine learning algorithm may use a reinforcement learning approach. In reinforcement learning, the algorithm can learn a policy of how to act given an observation of the world. Every action may have some impact in the environment, and the environment can provide feedback that guides the learning algorithm.

### System

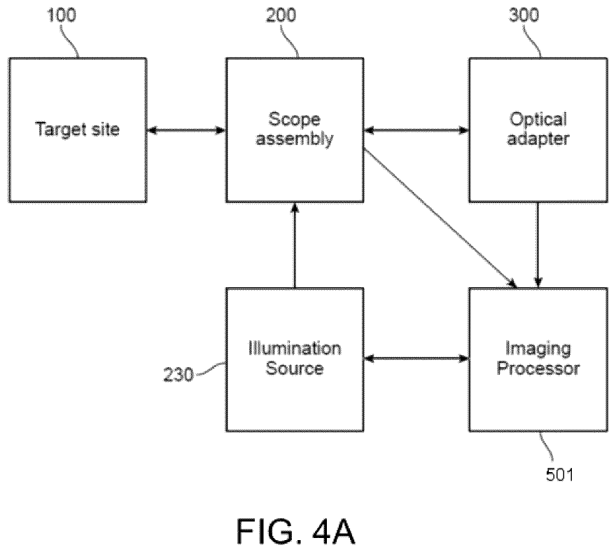

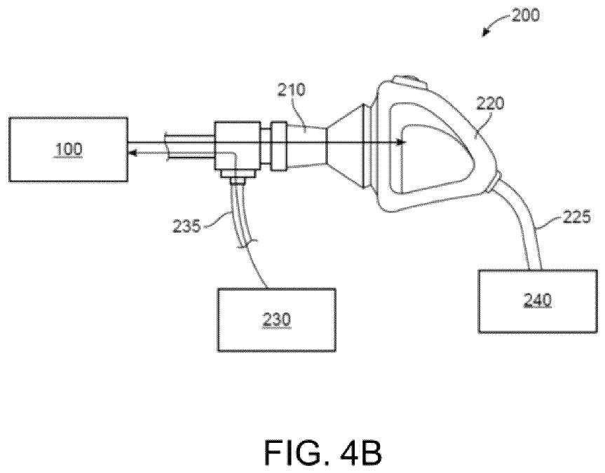

FIG. 4A schematically illustrates an example ecosystem for medical imaging. The ecosystem may comprise a target site 100 of a subject (e.g., a tissue site of interest of a patient, a surgical site, etc.). The ecosystem may comprise a scope assembly 200. The ecosystem may comprise an illumination source 230 in optical communication with the scope assembly 200. The illumination source 230 may be configured to provide one or more light beams (e.g., a combined light beam) via the scope assembly 200 and toward the target site 100. The target site 100 may be in optical communication with the scope assembly 200, such that (i) the target site 100 may be illuminated by the one or more light beams from the scope assembly 200 and (ii) the scope assembly 200 may detect one or more light signals reflected or emitted by the target site 100 upon such illumination. The scope assembly 200 may be configured to capture at least one image or video of the target site based on at least a portion of the one or more light signals from the target site 100. The ecosystem may comprise an optical adapter 300 that is operatively coupled to one or more components of the scope assembly 200. The optical adapter 300 may be in optical communication with the scope assembly 200, such that (i) the optical adapter 300 may receive one or more light signals from the scope assembly 200 and (ii) the scope assembly 200 may receive one or more light signals from the optical adapter 300. The optical adapter 300 may be configured to generate data (e.g., images, videos, lase speckle imaging, etc.) based on at least an additional portion of the one or more light signals from the target site 100. The generated data may encode different features of the target site than that of the at least one image or video captured by the scope assembly 200. The scope assembly 200 and the optical adapter 300 may be operatively coupled to an imaging processor 501. The imaging processor 501 may be configured to analyze or combine data, image(s), or video(s) generated by the scope assembly 200 and the optical adapter 300.

FIG. 4B schematically illustrates an example ecosystem of the scope assembly 200 in absence of the optical adapter 300. The scope assembly 200 comprises a scope 210 and a camera 220 that are operatively coupled to each other. The scope 210 and the camera 220 may me mechanically and optically in communication with each other. The scope 210 may be in optical communication with the illumination source 230 via an optical signal path 235 (e.g., an optical fiber). The illumination source 230 may direct one or more light beams via the optical signal path 235 and to the scope 210, and the scope 210 may direct the one or more light beams toward the target site 100. The scope 210 may also serve as an optical signal path for any light signals reflected or emitted by the target site 100 toward the camera 220. The camera 220 may be operatively coupled to the imaging processor 501 via a signal line 225 (e.g., electrical wire such as copper wire, optical fiber, etc.). In some cases, a focusing coupler may be disposed between the scope 210 and the camera 220. The focusing coupler may be permanently attached to the camera 220. The focusing coupler may comprise a focusing knob. Imaging processor 501 may comprise an example, variation, or embodiment of computer system 501 described herein.

Scope 210 may comprise may be configured to visualize external and/or inner surface of a tissue (e.g., skin or internal organ) of a subject. The scope may be used to (i) examine (e.g., visually examine) the tissue of the subject and (ii) diagnose and/or assist in a medical intervention (e.g., treatments, such as a surgery). In some cases, the scope may be an endoscope. Examples of the endoscope may include, but are not limited to, a cystoscope (bladder), nephroscope (kidney), bronchoscope (bronchus), arthroscope (joints) and colonoscope (colon), and laparoscope (abdomen or pelvis).

### Computer Systems

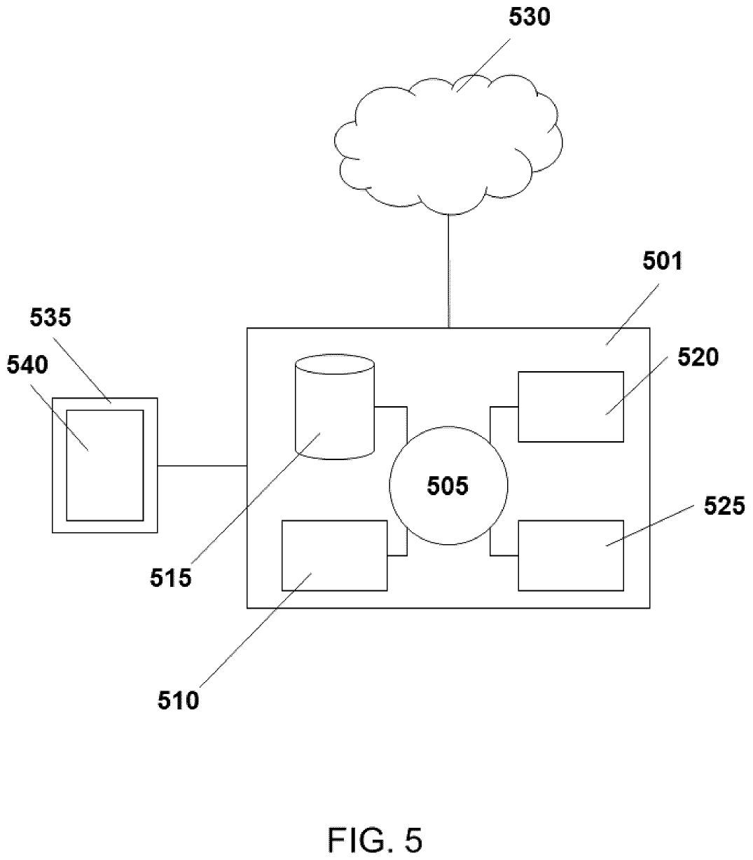

The present disclosure provides computer systems that are programmed or otherwise configured to implement methods of the disclosure, e.g., any of the subject methods for quantifying perfusion. FIG. 5 shows a computer system 501 that is programmed or otherwise configured to implement a method for quantifying perfusion. The computer system 501 may be configured to, for example, (a) control an imaging device to capture at least one image of a surgical scene; (b) process the at least one image to determine one or more perfusion characteristics associated with one or more reference regions in the surgical scene; and (c) determine one or more relative perfusion characteristics for a target region in the surgical scene based on the at least one image and the one or more perfusion characteristics associated with the one or more reference regions. In some cases, the computer system 501 can be an electronic device of a user or a computer system that is remotely located with respect to the electronic device. The electronic device can be a mobile electronic device.

The computer system 501 may include a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are located external to the computer system 501 (e.g., on a remote server that is in communication with the computer system 501 through an intranet or the Internet).

The computer system 501 can communicate with one or more remote computer systems through the network 530. For instance, the computer system 501 can communicate with a remote computer system of a user (e.g., a doctor, a surgeon, a medical worker, an imaging technician, etc.). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 501 via the network 530.

Methods as described herein can be implemented by way of machine (e.g., computer processor) executable code stored on an electronic storage location of the computer system 501, such as, for example, on the memory 510 or electronic storage unit 515. The machine executable or machine-readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that comprises a user interface (UI) 540 for providing, for example, a portal for a surgeon to (i) view one or more relative perfusion measurements for a target region (e.g., a tissue region) in a surgical scene or (ii) select one or more target regions of interest in order to view one or more relative perfusion measurements for the selected target regions of interest. The portal may be provided through an application programming interface (API). A user or entity can also interact with various elements in the portal via the UI. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 505. For example, the algorithm may be configured to (i) process at least one image of a surgical scene to determine one or more perfusion characteristics associated with one or more reference regions in the surgical scene, and (ii) determine one or more relative perfusion characteristics for a target region in the surgical scene based on the at least one image and the one or more perfusion characteristics associated with the one or more reference regions.

### EXAMPLES

The examples and embodiments described herein are for illustrative purposes only and are not intended to limit the scope of the claimed invention. Various modifications or changes in light of the examples and embodiments described herein will be suggested to persons skilled in the art and are to be include within the spirit and purview of this application and scope of the appended claims.

Methods: ActivSight™, an FDA-cleared device that displays both ICG and LSCI in minimally invasive surgery, was used to measure relative perfusion units (RPU) of a porcine intestine model. Perfusion quantification algorithms disclosed herein were used in concert with reference areas of normally perfused and ischemic tissue as disclosed here. To prepare the model, 1) selective devascularization of small bowel was performed to create a continuous gradient of perfused-watershed-ischemia; and 2) controlled occlusions were performed by outflow clamping of the aortic inflow/portal vein with arterial pressure monitoring in left iliac artery. RPUs were measured in three regions-perfused, watershed and ischemic segments. Positive and negative controls were mesenteric blood vessels and the avascular mesentery. Statistical analysis was performed by analysis of variance (ANOVA) and student's t-test.

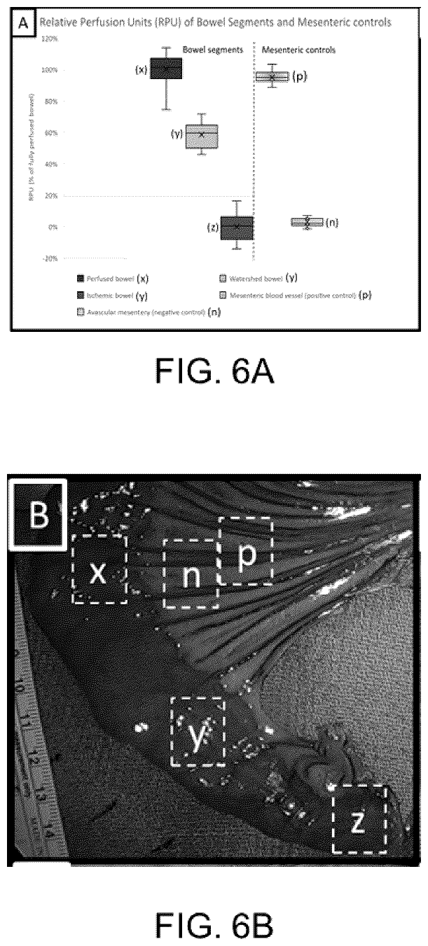

Results: FIG. 6A shows a box scatter plot with X-axis showing perfused (x), watershed (y), and ischemic (z) bowel segments. Also shown are positive (p) and negative (n) controls. FIG. 6B shows the regions x, y, z, p, and n in a representative porcine small intestine model. As shown in FIG. 6A, mean RPU measurements over the three bowel segments were able to clearly distinguish between the perfused (100%±10%), watershed (58%±8%) and ischemic (0%±8%) regions of small bowel (p<0.0001) with positive (95%±5%), and negative (3%±3%) mesenteric controls. Mean RPU of all three bowel segments were significantly different (p<0.0001). Mean RPU of perfused and ischemic bowel segments were not statistically different from positive (p=0.85) and negative (p=0.76) mesenteric controls, respectively. Measurements in mesenteric blood vessels and avascular mesentery were statistically equivalent to perfused and ischemic segments respectively.

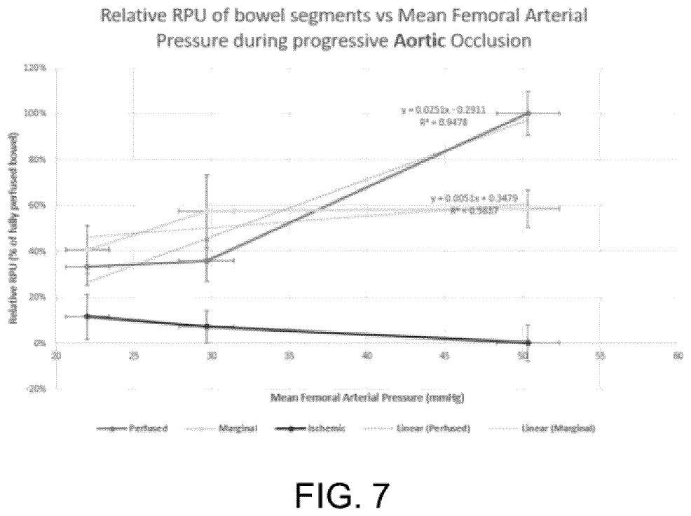

FIG. 7 shows a scatter plot of relative perfusion units of bowel segments versus the mean femoral arterial pressure during progressive aortic occlusion. As shown, with aortic occlusion, perfused bowel demonstrated strong linear correlation (r²=0.95) between RPU and mean arterial pressure (MAP), as did watershed bowel (r²=0.56) above a threshold MAP (37 mmHg). As shown, the ischemic region shows little change in RPU's with decreasing aortic occlusion (as measured by the mean femoral arterial pressure). As shown, the marginal region increases in perfusion with decreasing aortic occlusion; however, the relative perfusion never crosses above 60% of the perfusion in the fully perfused bowel. As shown, the perfused region increases in perfusion with decreasing aortic occlusion and increases to 100% of relative perfusion in the fully perfused bowel.

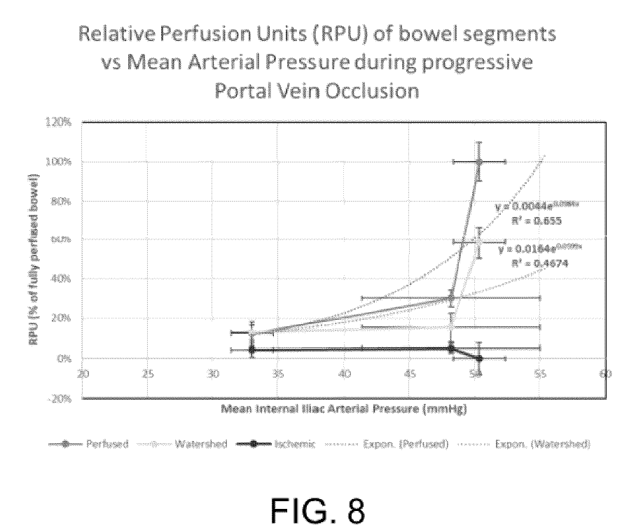

FIG. 8 shows a scatter plot of relative perfusion units of bowel segments versus the mean femoral arterial pressure during progressive portal vein occlusion. Strong positive non-linear correlations were observed between relative perfusion units (RPU) and mean arterial pressure (MAP) for perfused (Spearman correlation coefficient 0.81) and watershed bowel segments (Spearman correlation coefficient 0.68) in a model of venous occlusion. Intestinal RPU and perfusion colormap were highly sensitive to relatively small changes in MAP caused by venous occlusion (Perfused/Watershed=Mean RPU 100%±10%/58%±8% at MAP 50 mmHg vs. 30%±4%/15%±7% at MAP 48, p<0.00001). As shown, the ischemic region shows little change in RPU's with decreasing portal vein occlusion (as measured by the mean iliac arterial pressure). As shown, the marginal region increases in perfusion with decreasing portal vein occlusion;

however, the behavior non-linearly increases at about 48 mmHg going from about 15% to about 60%. As shown, the perfused region increases in perfusion with decreasing portal vein occlusion and increases to 100% of relative perfusion in the fully perfused bowel. Notably there is a large and non-linear increase at 48 mmHG mean internal iliac arterial pressure.

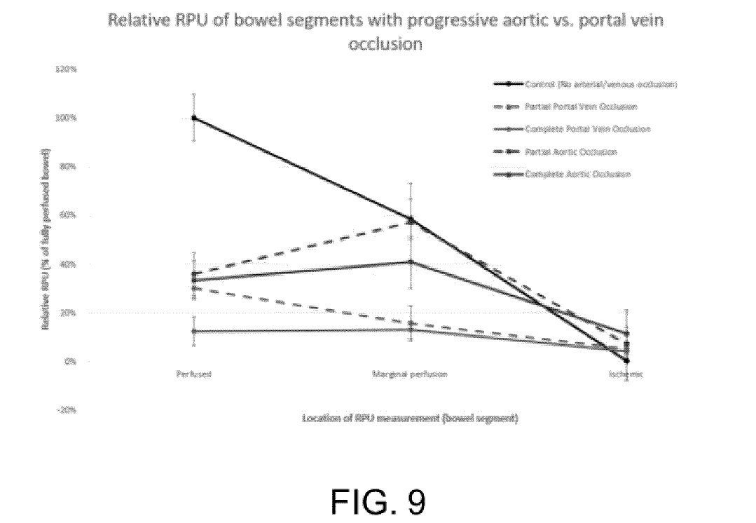

FIG. 9 shows a scatter plot of relative perfusion units of bowel segments with no arterial/venous occlusion vs progressive aortic occlusion vs progressive portal vein occlusion. On the x-axis, the position of the measurement along the perfused-ischemic gradient is indicated. On the y-axis, the RPU for each a control (top), partial aortic occlusion (dotted middle), complete aortic occlusion (solid middle), partial portal vein occlusion (dotted bottom), and complete portal vein occlusion (solid bottom).

Conclusion: The RPU metric, manifested through LSCI colormaps, was found to act as a measure of tissue perfusion/blood flow to distinctly detect perfused, watershed and ischemic regions in porcine intestine. RPU's were sensitive to real-time perfusion changes at tissue level with manipulation of arterial inflow and venous outflow. Perfusion changes resulting from arterial obstruction elicited linear RPU responses, while venous outflow obstruction induced non-linear RPU and colormap changes. Intestinal tissue perfusion measurements appeared to be more sensitive to venous outflow obstruction and may serve as a real-time, dye-free tool for intestinal anastomotic assessment.

Without being limited by theory, the difference in linearity between inflow and outflow perfusion may relate to the elasticity of the small bowel. For example, when the outflow is clamped, the small bowel may expand under the pressure of the blood flowing into the bowel. The expansion of the organ may be limited. Thus, for lower amounts of clamping, pressure in the bowel may be slower to rise. For higher amounts of clamping, the pressure may increase more quickly as the bowel reaches the limit of its expansion.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1. A method for quantifying perfusion, comprising:
(a) obtaining at least one image of a surgical scene;
(b) processing the at least one image to determine one or more perfusion characteristics associated with one or more reference regions in the surgical scene; and
(c) determining one or more relative perfusion characteristics for a target region in the surgical scene based on a ratio of one or more perfusion characteristics the target region the one or more perfusion characteristics associated with the one or more reference regions.

2. The method of claim 1, further comprising distinguishing an etiology of tissue ischemia as an inflow obstruction or an outflow obstruction based on the one or more relative perfusion characteristics.

3. The method of claim 1, wherein at least one of the reference regions is used as a negative control representing an ischemic reference region.

4. The method of claim 1, wherein at least one of the reference regions is used as a positive control representing a perfused reference region.

5. The method of claim 1, wherein (b) further comprises using a linear relationship between the one or more reference regions to determine the one or more relative perfusion characteristics.

6. The method of claim 5, wherein the linear relationship relates a laser speckle contrast value to the one or more relative perfusion characteristics.

7. (canceled)

8. The method of claim 1, further comprising differentiating between the one or more reference regions based at least in part on pulsatility behavior referenced numbers.

9. The method of claim 8, wherein the differentiating is based at least in part on one or more colormaps comprising laser speckle data.

10. (canceled)

11. (canceled)

12. The method of claim 1, wherein the at least one image comprises at least one member selected from the group consisting of: a laser speckle image, a time-of-flight image, one or more multispectral images, and a fluorescence image.

13. (canceled)

14. (canceled)

15. (canceled)

16. The method of claim 1, wherein the at least one image comprises at least a laser speckle image and at least one image of a second image type, wherein the second image type is selected from the group consisting of: a time-of-flight image, one or more multispectral images, and a fluorescence image.

17. (canceled)

18. (canceled)

19. (canceled)

20. (canceled)

21. (canceled)

22. (canceled)

23. (canceled)

24. (canceled)

25. The method of claim 1, further comprising using one or more time-of-flight measurements to standardize perfusion quantification independent of camera positioning.

26. (canceled)

27. (canceled)

28. (canceled)

29. A system for medical imaging, the system comprising:
processor operably connected to a non-transitory computer readable storage medium with instructions stored thereon, wherein the processor is configured to implement the instructions to:
(a) obtain at least one image of a surgical scene;
(b) process the at least one image to determine one or more perfusion characteristics associated with one or more reference regions in the surgical scene; and
(c) determine one or more relative perfusion characteristics for a target region in the surgical scene based on a ratio of one or more perfusion characteristics of the target region to the one or more perfusion characteristics associated with the one or more reference regions.

30. The system of claim 0, wherein the processor is further configured to determine an etiology of tissue ischemia as an inflow obstruction or an outflow obstruction based on the one or more relative perfusion characteristics.

31. (canceled)

32. (canceled)

33. The system of claim 0, wherein at (b) the processor is further configured to use a linear relationship between the one or more reference regions; to determine the one or more relative perfusion characteristics.

34. The system of claim 33, wherein the linear relationship relates a laser speckle contrast value to the one or more relative perfusion characteristics.

35. (canceled)

36. The system of claim 29, wherein the processor is further configured to differentiate between the one or more reference regions based at least in part on pulsatility behavior referenced numbers.

37. The system of claim 36, wherein the differentiating is based at least in part on one or more colormaps comprising laser speckle data.

38. (canceled)

39. (canceled)

40. The system of claim 0, wherein the at least one image comprises at least one member selected from the group consisting of: a laser speckle image, a time-of-flight image, one or more multispectral images, and a fluorescence image.

41. (canceled)

42. (canceled)

43. (canceled)

44. The system of claim 0, wherein the at least one image comprises at least a laser speckle image and at least one image of a second image type, wherein the second image type is selected from the group consisting of: a time-of-flight image, one or more multispectral images, and a fluorescence image.

45. (canceled)

46. (canceled)

47. (canceled)

48. (canceled)

49. (canceled)

50. (canceled)

51. (canceled)

52. (canceled)

53. The system of claim 0, wherein the processor is further configured to use one or more time-of-flight measurements to standardize perfusion quantification independent of camera positioning.

54. (canceled)

55. (canceled)

56. (canceled)
